(19) United States
(12) Patent Application Publication
(10) Pub. No.: US 2023/0288337 A1
(43) Pub. Date: Sep. 14, 2023

(54) HIGH DIMENSIONAL FINGERPRINTS OF SINGLE NANOPARTICLES AND THEIR USE IN MULTIPLEXED DIGITAL ASSAYS

(71) Applicant: University of Technology Sydney, Ultimo, New South Wales (AU)

(72) Inventors: Dayong JIN, Ultimo, New South Wales (AU); Jiajia ZHOU, Ultimo, New South Wales (AU); Jiayan LIAO, Ultimo, New South Wales (AU)

(21) Appl. No.: 18/040,513

(22) PCT Filed: Aug. 4, 2021
(86) PCT No.: PCT/AU2021/050849

§ 371 Date: Feb. 3, 2023

(30) Foreign Application Priority Data
Aug. 4, 2020 (AU) 2020902731

(51) Int. Cl. G01N 21/64; C09K 11/77; C12Q 1/6816

(52) U.S. Cl. CPC G01N 21/6408; C09K 11/7773; G01N 21/6458; C12Q 1/6816; B82Y 15/00

(57) ABSTRACT

Methods are presented for tuning the time-domain emissive profile of single upconversion nanoparticles using a number of different techniques so as to increase the coding capacity at the nanoscale. The disclosure also relates to time-resolved wide-field imaging and deep-learning techniques to decode the nanoparticle fingerprints.

### FIELD OF THE DISCLOSURE

The present disclosure relates generally to methods for tuning the time-domain emissive profile of single upconversion nanoparticles using a number of different techniques so as to increase the coding capacity at the nanoscale. The disclosure also relates to time-resolved wide-field imaging and deep-learning techniques to decode the nanoparticle fingerprints.

### BACKGROUND OF THE DISCLOSURE

Any discussion of the prior art throughout this specification should in no way be considered as an admission that such prior art is widely known or forms part of the common general knowledge in the field.

It is the ultimate goal of nanotechnology to manipulate structures with unprecedented accuracy and to tune their functions to precisely match the parameters required at the single nanoparticle level. Optical multiplexing with increased capacity will advance the ongoing development of next-generation enabling technologies, spanning from high-capacity data storage, anti-counterfeiting, large-volume information communication, to high-throughput screening of multiple single molecular analytes in a single test and super-resolution imaging of multiple cellular compartments.

Super-capacity optical multiplexing challenges our ability in creating multiplexed codes in orthogonal dimensions, e.g. intensity, colour, polarization and decay time, assigning them to the microscopic and nanoscale carriers, and decoding them in high throughput fashion with sufficient accuracy in the orthogonal optical dimensions. Though desirably the size of material that carries the optical barcodes can be pushed from microscopic to the nanoscopic range, it sacrifices the overall amount of emissive photons (brightness), and therefore limits the number of detectable codes, e.g. typically three to four colour channels or brightness levels. The amount of signal emitted from a nanoscale object can drop exponentially and their size is often below the optical diffraction limit. This prevents the conventional filter optics and detection process from decoding them with sufficient spectral-spatial resolutions.

This unmet need poses significant challenges for material sciences to pursue fabrication strategies and the precise control in producing uniform nanoscopic carriers, and further challenges the photonics community to maximize the number of emissive photons and to explore the diversity of optical information that can be produced in multiple orthogonal dimensions, such as emission colours (spectrum), lifetime, polarization and angular momentum.

Lanthanide-doped upconversion nanoparticles (UCNPs) absorb low-energy near-infrared photons to emit high-energy emissions in the visible and UV regions. Single UCNPs are uniform, photo-stable for hours and allow single nanoparticle tracking experiments in live cells. Recently, the core-shell-shell design of each single UCNP has been reported as emitting ˜200 photons per second under a low irradiance of 8 W/cm³, and intensity uniform UCNPs have enabled the single-molecule (digital) immuno assay. The colour-based multiplexing of UCNPs can be realized by tuning the dopants, core-shell structure or excitation pulse durations, but all colour-based approaches are intrinsically limited by cross-talk in the spectrum domain. Major advances have been made in the ensemble lifetime measurements of microsphere arrays, time-domain contrast agents for deep-tissue tumour imaging and high-security-level anticounterfeiting applications. Though lifetime multiplexing with single nanoparticle sensitivity was possible, the relatively low brightness and point scanning confocal microscopy have limited the readout throughput.

### SUMMARY OF THE DISCLOSURE

In a first aspect the present disclosure provides a method for tuning a time-domain emissive profile of an upconversion nanoparticle, the method comprising the step of manipulating a rising, decay and/or peak moment of an excited state population.

Manipulation of the rising, decay and/or peak moment of the excited state population may be achieved by altering interfacial energy migration in the nanoparticles.

Interfacial energy migration may be altered by exposing the nanoparticle to different excitation wavelengths.

The nanoparticles may be UCNPs.

The UCNPs may comprise one or more of: neodymium, ytterbium, thulium, erbium, lanthanum, cerium, praseodymium, neodymium, promethium, samarium, europium, gadolinium, terbium, dysprosium, holmium, lutetium, scandium and yttrium.

The UCNPs may comprise neodymium, ytterbium, thulium and/or erbium.

The UCNPs may contain a host material selected from an alkali fluoride, an oxide or an oxysulfide.

The alkali fluoride may be NaGdF₄, Ca₂F, NaYF₄, LiYF₄, NaLuF₄ or LiLuF₄, KMnF₃, and the oxide may be Y₂O₃. Mixtures of these materials are also contemplated. In one embodiment, the host material is NaYF₄.

Where the UCNPs are crystalline, the NaYF₄ may be hexagonal phase, or any other crystal phase.

The UCNPs may be core-multi-shell UCNPs.

The core-multi-shell UCNPs may comprise a core, a migration layer and a sensitisation layer.

The migration layer may comprise Yb³⁺.

The sensitization layer may comprise Yb³⁺ and Nd³⁺.

The core may comprise Yb³⁺, Er³⁺ and/or Tm³⁺.

The core may comprise Yb³⁺ and Er³⁺ or Yb³⁺ and Tm³⁺.

The UCNPs may be selected from: corea-multi-shell β-NaYF₄: Nd³⁺, Yb³⁺, Tm³⁺ UCNPs and core-multi-shell β-NaYF₄: Nd³⁺, Yb³⁺, Er³⁺ UCNPs.

The UCNPs may have a coefficient of variation (CV) value less than about 15%, or less than about 10%, or less than about 5%.

In a second aspect the present invention provides a multiplex assay method for identifying a luminescent probe in a multiplex assay, the method comprising: stimulating the luminescent probe to produce luminescence, and measuring the rising time, peak moment and/or decay time of the luminescence.

The multiplex array may be a suspension array.

The method may further comprise: stimulating a plurality of luminescent probes to produce luminescence; measuring the rising times, peak moments and/or decay times of the luminescence, and identifying one or more probes based on differences in the rising times, peak moments and/or decay times.

The rising time, peak moment and/or decay time of the luminescence may provide one or more codes.

The luminescent probe may be a nano-tag, sphere, particle or carrier.

The luminescent probe may include one or more nanoparticles.

The luminescent probe may include one or more nanoparticles as described above in connection with the first aspect.

In a third aspect the present invention provides a method for performing a multiplex assay, the multiplex assay including using, as probes, a plurality of nanoparticles having luminescence profiles possessing different rising times, peak moments and/or decay times, wherein the probes are distinguished from one another based on their differing rising times, peak moments and/or decay times.

The luminescent probe may include one or more nanoparticles as described above in connection with the first aspect.

In a fourth aspect the present invention provides a method for preparing a library of spectrally distinct nanoparticles comprising:
(a) providing a plurality of different classes of nanoparticles, wherein each different class of nanoparticle has a luminescence profile possessing distinct rising times, peak moments and/or decay times;
(b) varying one or more of the following parameters of the nanoparticles within each class, so as to provide the library of spectrally distinct nanoparticles:
   core size of the nanoparticles;
   concentrations of emitter ions and sensitiser ions in the core;
   thickness of a sensitisation layer;
   concentration of sensitiser ions in the sensitisation layer; and
presence or absence of a passivation layer.

In one embodiment, at least three different classes of nanoparticles are prepared, and each class comprises at least 10 different types of nanoparticles.

The nanoparticles may be UCNPs.

The different classes of UCNPs may be classes of UCNPs having different combinations of activators and/or sensitisers.

The UCNPs may comprise one or more of: neodymium, ytterbium, thulium, erbium, lanthanum, cerium, praseodymium, neodymium, promethium, samarium, europium, gadolinium, terbium, dysprosium, holmium, lutetium, scandium and yttrium.

The UCNPs may comprise neodymium, ytterbium, thulium and/or erbium.

The UCNPs may contain a host material selected from an alkali fluoride, an oxide or an oxysulfide.

The alkali fluoride may be NaGdF₄, Ca₂F, NaYF₄, LiYF₄, NaLuF₄ or LiLuF₄, KMnF₃, and the oxide may be Y₂O₃. Mixtures of these materials are also contemplated. In one embodiment, the host material is NaYF₄.

Where the UCNPs are crystalline, the NaYF₄ may be hexagonal phase, or any other crystal phase.

In one embodiment, the plurality of different classes of UCNPs includes at least one class having core-multi-shell UCNPs.

The core-multi-shell UCNPs may comprise a core, a migration layer and a sensitisation layer.

The migration layer may comprise Yb³⁺.

The sensitization layer may comprise Yb³⁺ and Nd³⁺.

The core may comprise Yb³⁺, Er³⁺ and/or Tm³⁺.

The core may comprise Yb³⁺ and Er³⁺ or Yb³⁺ and Tm³⁺.

In one embodiment, the plurality of different classes of UCNPs includes the following: core-multi-shell β-NaYF₄: Nd³⁺, Yb³⁺, Tm³⁺ UCNPs, core-multi-shell β-NaYF₄: Nd³⁺, Yb³⁺, Er³⁺ UCNPs and β-NaYF₄: Yb³⁺, Tm³⁺ UCNPs.

The UCNPs may have a coefficient of variation (CV) value less than about 15%, or less than about 10%, or less than about 5%.

In one embodiment, all of the parameters are varied.

In a fifth aspect the present invention provides a library of spectrally distinct nanoparticles when obtained by the method of the fourth aspect.

In a six aspect the present invention provides use of the library of spectrally distinct nanoparticles of the fifth aspect in a multiplex assay, wherein the nanoparticles are used as probes.

The probes may be distinguished from one another based on at least differing rising times, peak moments and/or decay times of their luminescence profiles.

The probes may be decoded using wide-field time-resolved microscopy or deep learning.

### BRIEF DESCRIPTION OF THE DRAWINGS

FIG. 1: Creation of monodisperse UCNPs with optical information in orthogonal dimensions (a) TEM image of a kind of typical morphology uniform core-shell nanoparticles β-NaYF₄: Yb³⁺, Tm³⁺. (b and c) Ensemble upconversion emission spectrum (b) and lifetime profile (c) of the Yb³⁺, Tm³⁺ doped UCNPs under 976 nm excitation. (d) HADFF-STEM observation showing the core-multi-shell structure of the nanoparticles doped with Nd³⁺, Yb³⁺, Er³⁺ in different layers. (e) Energy level diagram of core-multi-shell nanoparticles showing the cascade photon energy sensitization, transfer and conversion process: Nd³⁺ sensitization at 808 nm, Yb³⁺-mediated interfacial energy migration (IEM) at 976 nm, and upconversion of near-infrared photons into higher-energy visible emissions in a typical Yb³⁺—Er³⁺ system. (f and g) Ensemble upconversion emission spectra (f) and lifetime curves (g) of Nd³⁺, Yb³⁺, Er³⁺ doped UCNPs under 808 nm and 976 nm excitations.

FIG. 2: Time-domain τ² profile control through upconversion energy transfer schemes and materials engineering (a) Illustrations of five strategies used for τ² profile tuning, i.e. core size, the concentrations of sensitizers and emitters in the core, the sensitization layer thickness, the concentration of sensitizers in the sensitization layer, and the passivation layer. (b-d) τ² profile tuning of three series of samples, i.e., Yb—Tm series (b), Nd—Yb—Tm series (c), and Nd—Yb—Er series (d), under NIR excitation. Dot lines indicate the normalized intensity of 1/e (I_{1/e}). (e-g) Calculated rising time (τ_{I}_{₁}−τ_{I}_{_{1/e−}}rising), peak moment (τ_{I}_{₁}), and decay time (τᵢ_{_{1/e−}}decay−τ_{I}_{₁}) according to the curves in panels (b-d) for Yb—Tm series (e), Nd—Yb—Tm series (f), and Nd—Yb—Er series (g). (h-j) Photos of representative UCNPs in Yb—Tm series (h), Nd—Yb—Tm series (i), and Nd—Yb—Er series (j) showing their upconversion colours under NIR excitation.

FIG. 3: Confocal and wide-field characterization of τ²-Dots (a-d) Confocal microscopic single nanoparticle imaging (a), brightness distribution (b), the long-term photostability of a single dot (c) under 808 nm CW excitation at 5.5×10⁶ W/cm², and corresponding lifetime curves (d) of single dots 1-6 in (e) under 808 nm pulse excitation (by modulating the CW laser at 5.46 kW/cm²). (e) Schematic illustration of the transient fluorescence signal detection principle using a time-resolved sCMOS camera for wide-field microscopy. (f and g) A comparison of the time-resolved 6^{th}, 16^{th}, 28^{th} and 48^{th} frames of τ²-Dots-stained micro-polystyrene beads (f) and single τ²-Dot (g) within a beam area of 28 μm in diameter. (h) Lifetime curve of a single τ²-Dots-stained bead, which is indicated by a dotted square in (f). (i) Lifetime curves of a single and ensemble of τ²-Dots, which are indicated by yellow and dark orange dotted squares in (g). All the data associate with a random batch of τ²-Dot (τ²−13).

FIG. 4: Time-domain optical fingerprints from fourteen batches of τ²-Dots (a) Lifetime curve statistics from single τ²-Dots. Shaded areas cover the lifetime curves of more than 20 single dots from each type of τ²-Dots. The solid colourful lines represent the averaged lifetime curves for each type of τ²-Dots. (b and e) Intensity normalized display of averaged single nanoparticle lifetime fingerprints of Yb—Tm series (nine) τ²-Dots and Nd—Yb—Er series (five) τ²-Dots. (c and f) The histograms of single-particle decay indicator (to) distribution analysis for the nine batches of Yb—Tm samples τ²-1 to τ²-9 (c) and the four batches of Nd—Yb—Er samples τ²-10 to τ²-14 (f). (d and g) Scatter plots of decay and rising indicators (τ_{D} and τ_{R}) of samples τ²-1 to τ²-9 (d) and τ²-10 to τ²-14 (g). Both the indicators (τ_{D} and τ_{R}) are defined as the time moment at 1/e of the maximum intensity.

FIG. 5: Deep learning aided decoding of the fingerprints of single τ²-Dots (a) Illustration of the neural network used for the classification task. (b and c) Classification result for the Yb—Tm series τ²-1 to τ²-9 dots (b) and Nd—Yb—Er series τ²-10 to τ²-14 dots (c) (for visualization purpose, pseudocolour is used to represent each type of single dots). (d and e) Mean classification accuracy obtained through cross-validation with the database of 6 training sets and 1 validation set for each type of dots.

FIG. 6: Demonstration of the potentials of using the library of single τ²-Dots' optical fingerprints for a diverse range of applications (a) Time-domain anti-counterfeiting by using three types of τ²-Dot security inks with different rising-decay fingerprints. (b) Multiplexed single molecule digital assays using five types of τ²-Dot probes to quantify the five target pathogen single-strand DNAs (HBV, HCV, HIV, HPV-16, and EV). The cartoon illustration showing the probe-DNA conjugation procedure on a 96 well plate. (c) Three types of τ²-Dots resolved by upconversion structure illumination microscopy (U-SIM).

FIG. 7: SEM images of microbeads. SEM photos of 5 μm polystyrene beads before (a) and after (b) tagged with τ²-13 nanoparticles. Scale bars: 1 μm.

FIG. 8: Correlated wide-field optical image and SEM image of τ²-13 Dots, confirming the single particle nature. (a) wide-field optical image under 808 nm laser, (b) the corresponding SEM image of the same area.

FIG. 9: Schematic view of confocal microscopy. (SMF, single-mode fiber; MMF, multi-mode fiber; L1, collimation lens; L2, collection lens; HWP, half-wave plate; PBS, polarized beam splitter; FM, flexible mirror; DM, dichroic mirror; Obj, objective lens; SPF, short pass filter; SPAD, single-photon avalanche diode; CCD, charge-coupled device).

FIG. 10: Schematic view of wide-field fluorescence imaging setup. (SMF, single-mode fiber; MMF, multi-mode fiber; L1 &L6: collimation lens; L2 and L3 & L7 and L8: lenses for beam expanding; L4 & L9: tube lenses; DM1&DM2: dichroic mirrors; Obj: objective lens; L5: collection lens; SPF: short pass filter; FM, flexible mirror).

FIG. 11: Time-resolved structured illumination microscopy for sub-diffraction imaging. SMF, single-mode fiber; L1: collimation lens; L2 and L3: lenses for beam expanding; M: silver mirror; DMD: digital micromirror device; L4-L6: relay lens; DM: dichroic mirror; Obj: objective lens; L7: collection lens; SPF: short pass filter).

FIG. 12: TEM photos and size histograms of Yb—Nd—Tm series samples (15-26). Scale bars: 100 nm

FIG. 13: TEM photos and size histograms for Yb—Nd—Er series samples (27-42). Scale bar: 200 nm

FIG. 14: TEM photos and size histograms of the Yb—Tm series samples (1-14). Scale bars: 200 nm

FIG. 15: Confocal microscopy images and statistical intensities of Nd—Yb—Er τ²-Dots. Confocal microscopy quantitative measurement of the whole spectrum luminescence emission of Nd—Yb—Er τ²-Dots under 808 nm excitation at the power density of 5.5×10⁶ W/cm². Scale bar: 1 μm.

FIG. 16: The power-dependent curve of single nanoparticle brightness collected by a SPAD for τ²-10 under 808 nm excitation. The two dotted lines show the emission intensities under power densities of 5.5×10³ W/cm² (for wide-field imaging) and 7.6×10⁶ W/cm² (for confocal imaging).

FIG. 17: Simulated excitation field under wide-field microscopy. The pattern is a two-dimensional gaussian shape with a spot size of 29.89 μm in x and 28.44 μm in y, measured from the fitting of emission mapped pattern.

FIG. 18: Excitation power dependence of Nd—Yb—Er τ²-Dots. Laser power dependence of the upconverted emissions of whole spectra region of Nd—Yb—Er τ²-Dots samples under wide-field microscopy.

FIG. 19: The decay time histograms of τ²-Dots. The numeral beside each histogram is the mean decay time±decay time CV under wide-field microscopy. The lifetime imaging sequences were acquired under the 808 nm excitation pulse laser of 0-200 μs.

FIG. 20: τ² profile similarity of different samples. (a) Lifetime curves of τ²-1 and τ²-2 and (b) Lifetime curves of τ²-11 and τ²-12, showing the lifetime fingerprints highly overlap with each other.

FIG. 21: (a) Mean classification accuracies of 7 batches of Yb—Nd—Er τ²-Dots samples after 50 times randomly cross-validation. (b) Single nanoparticle intensities under the wide-field microscopy with the same imaging condition of above 7 τ²-Dots. The averaged brightness was achieved based on counting more than 100 nanoparticles. (c) Lifetime curve statistics from more than 20 single nanoparticles of sample 40. When training 7 batches of UCNPs by adding the sample 40 that has relatively weak emission intensity, the classification accuracies of these 7 samples are around 90%. Meanwhile, the classification accuracy of sample 40 is the lowest.

### DETAILED DESCRIPTION

In the context of this specification the term “about” is understood to refer to a range of numbers that a person of skill in the art would consider equivalent to the recited value in the context of achieving the same function or result.

In the context of this specification the terms “a” and “an” are used herein to refer to one or to more than one (i.e. to at least one) of the grammatical object of the article. By way of example, “an element” means one element or more than one element.

Throughout this specification, unless the context requires otherwise, the word “comprise”, or variations such as “comprises” or “comprising” will be understood to imply the inclusion of a stated element, integer or step, or group of elements, integers or steps, but not the exclusion of any other element, integer or step, or group of elements, integers or steps. Thus, in the context of this specification, the term “comprising” means “including principally, but not necessarily solely”.

The present inventors have discovered that the time-domain emissive profile from single upconversion nanoparticles, including the rising, decay and peak moment of the excited state population (τ² profile) can be arbitrarily tuned by upconversion schemes, including interfacial energy migration, concentration dependency, energy transfer, and isolation of surface quenchers. This allows a significant increase in the coding capacity at the nanoscale. It has also been found that at least three orthogonal dimensions, including the excitation wavelength, emission colour and τ² profile, can be built into the nanoscale derivative τ²-dots. These high-dimensional optical signatures can be pre-selected to build a vast library of single-particle nano-tags. These high-dimensional optical fingerprints provide a new horizon for applications spanning from sub-diffraction-limit data storage, security inks, to high-throughput single-molecule digital assays and super-resolution imaging.

### Control of τ² Profile and the Role of Interfacial Energy Migration in Life Time Engineering

The applicant has demonstrated that the morphology of both active core @ inert shell UCNPs and active core @ energy migration shell @ sensitization shell @ inert shell UCNPs can be highly controlled, and that once the single UCNP is sufficiently bright under wide-field microscopy it displays its characteristic optical signatures in the time domain. Surprisingly, not only is the decay time of each batch of UCNPs tunable, but also the rising time, decay time and peak moment of the excited state population from a single nanoparticle. The inventors have found that the rising time, decay time and peak moment can be further manipulated by a multi-interfacial energy transfer process and orthogonal excitation wavelengths. Accordingly, in one aspect the present invention provides a method for tuning a time-domain emissive profile of an upconversion nanoparticle, the method comprising manipulation of a rising, decay and/or peak moment of an excited state population. In one embodiment, manipulation of the rising, decay and/or peak moment of the excited state population may be achieved by altering interfacial energy migration (IEM) in the nanoparticles. In one embodiment, IEM may be altered by exposing the nanoparticle to different excitation wavelengths.

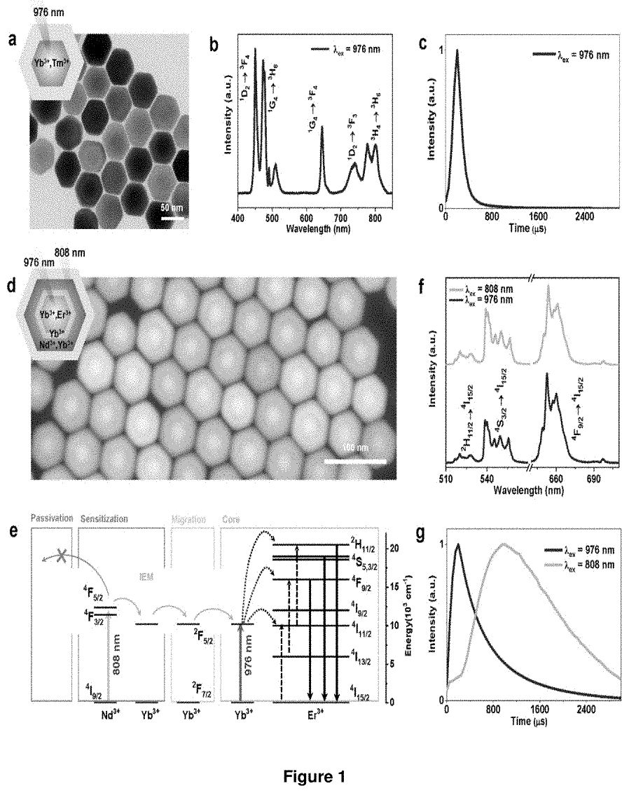

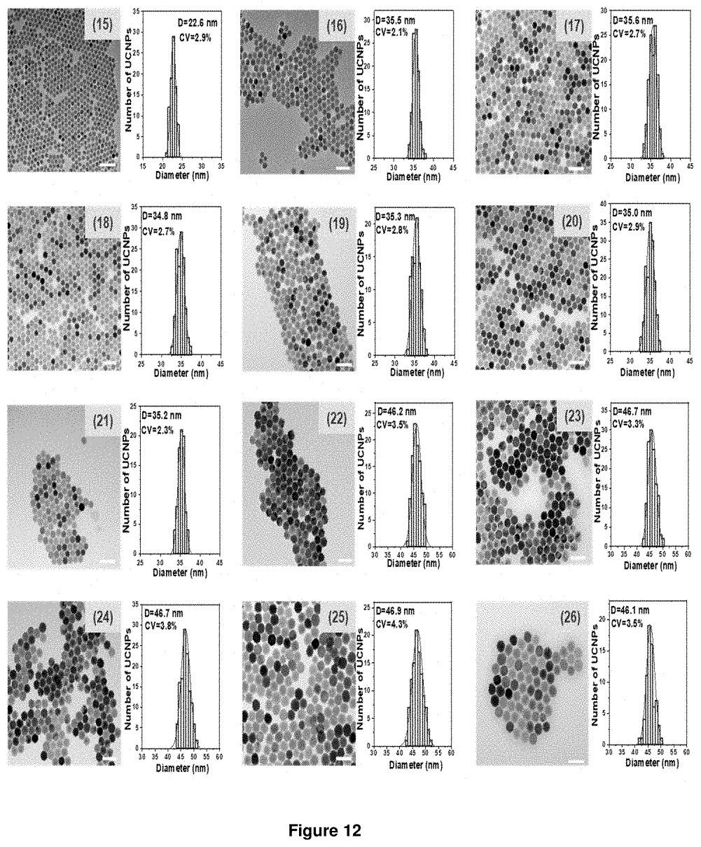

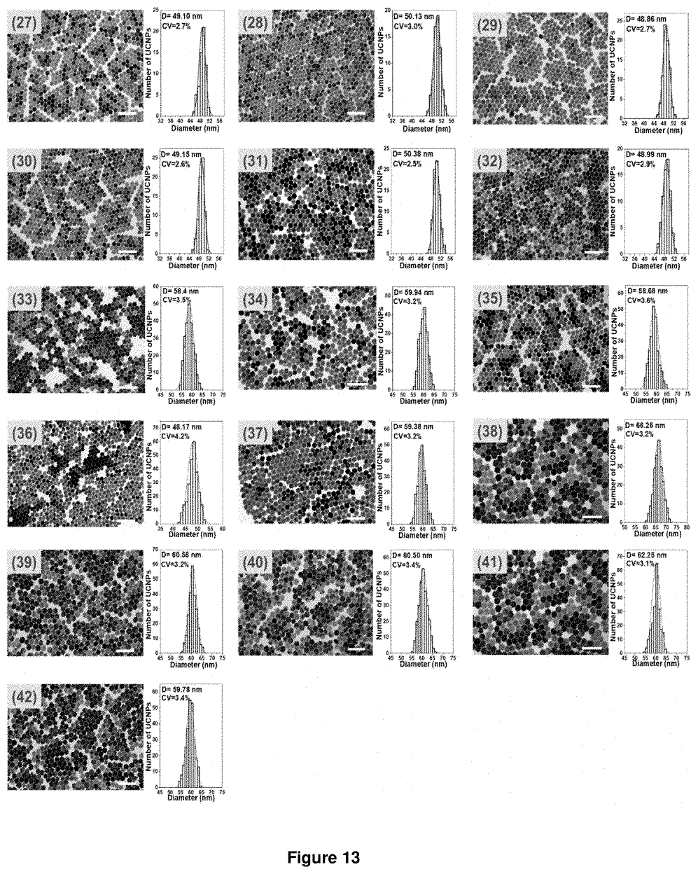

To demonstrate the role of IEM in manipulating the rising, decay and/or peak moment of an excited population, a series of core-multi-shell β-NaYF₄: Nd³⁺, Yb³⁺, Tm³⁺ UCNPs (FIG. 12) and β-NaYF₄: Nd³⁺, Yb³⁺, Er³⁺ UCNPs (FIG. 1d and FIG. 13) with a morphology uniformity (CV<5%) were prepared. The sophisticated design of core-multi-shell UCNPs permits an arbitrary control in the energy transfer process within a single nanoparticle as illustrated in FIG. 1e: the shell co-doped with Nd³⁺ and Yb³⁺ ions sensitizes 808 nm excitation, the energy migration shell containing a small percentage of Yb³⁺ ions is responsible for passing on the absorbed energy to the conventional Yb³⁺, Er³⁺ co-doped core that emits up-converted emissions at green and red bands (see FIG. 1f), and an inert shell is employed to prevent the energy migration to the surface quenchers, as well as to improve the optical uniformity of single nanoparticles. The multiple shells can significantly slow down the interfacial energy migration (IEM) process from primary sensitizer Nd³⁺ to the secondary sensitizer Yb³⁺ under the excitation of 808 nm. IEM plays an important role in the slow accumulation of the excited state populations, displayed as a time-delayed up-rising curve of upconversion emissions. To verify this IEM effect, the Yb³⁺ and Nd³⁺ ions were selectively excited using 976 nm and 808 nm lasers, respectively, and observed the same emission spectra (FIG. 1f). However, significant differences in the τ² profiles were observed (FIG. 1g). In this regard, the rising time for the Er³⁺ excited state populations to reach plateau is prolonged from 200 μs to 950 μs when the IEM process is involved.

The ability to tune the rising time, decay time and peak moment opens the possibility for this dimension to be used in multiplexing assays. Accordingly, in another aspect the present invention provides a multiplex assay method for identifying a luminescent probe in a multiplex assay, the method comprising: stimulating the luminescent probe to produce luminescence, and measuring the rising time, peak moment and/or decay time of the luminescence. In a further aspect the present invention provides a method for performing a multiplex assay, the multiplex assay including using, as probes, a plurality of nanoparticles having luminescence profiles possessing different rising times, peak moments and/or decay times, wherein the probes are distinguished from one another based on their differing rising times, peak moments and/or decay times.

### Orthogonal Optical Fingerprint Encoding

By harnessing the ability to tune the τ² profile of UCNPs, the applicant has created a set of time-domain optical fingerprints and built a library of different batches of τ²-Dots by implementing five strategies to tailor the excited-state populations of emitters present in the UCNPs. Accordingly, in a further aspect the present invention provides a method for preparing a library of spectrally distinct nanoparticles comprising:
(a) providing a plurality of different classes of nanoparticles, wherein each different class of nanoparticle has a luminescence profile possessing distinct rising times, peak moments and/or decay times;
(b) varying one or more of the following parameters of the nanoparticles within each class, so as to provide the library of spectrally distinct nanoparticles:
   core size of the nanoparticles;
   concentrations of emitter ions and sensitiser ions in the core;
   thickness of a sensitisation layer;
   concentration of sensitiser ions in the sensitisation layer; and
   presence or absence of a passivation layer.

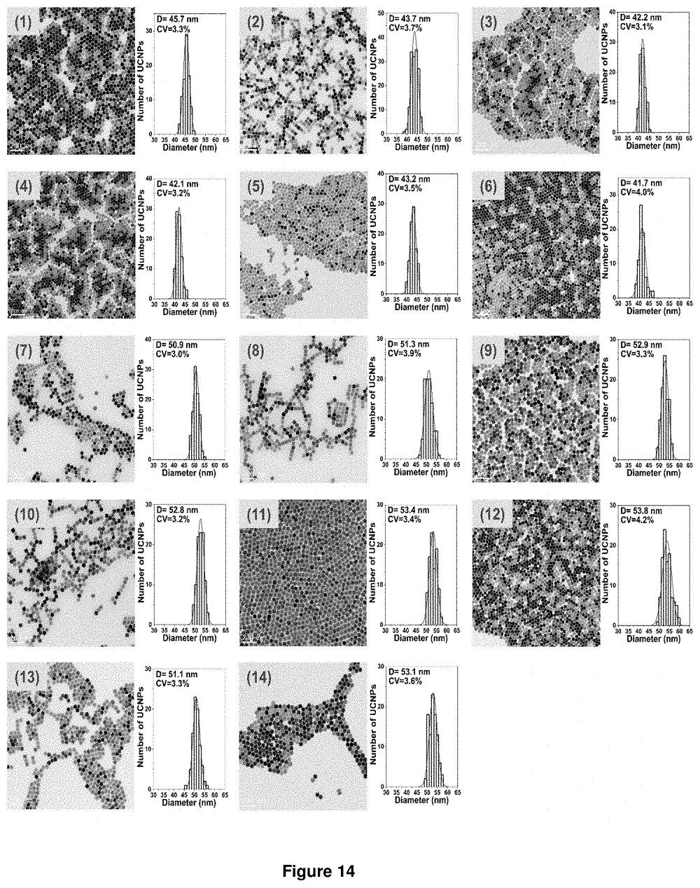

The exemplary library is based on three series of UCNPs as set out in Table 1, displaying three orthogonal dimensions (excitation wavelength, emission wavelength, and lifetime) of optical fingerprints. The Yb—Tm series (FIG. 1a-1c) can be excited at 976 nm, the Nd—Yb—Er series (FIG. 1d-1g) and Nd—Yb—Tm allow both 976 nm and 808 nm laser excitations. The TEM images in FIG. 1a and FIG. 14 shows the uniform spherical β-NaYF₄: Yb³⁺, Tm³⁺ core @ inert shell nanoparticles (coefficients of variation (CV)<5%). Upon excitation of 976 nm, the nanoparticles emit in blue, red and near-infrared (NIR) spectral bands, which are assigned to the diverse transitions of Tm³⁺ (FIG. 1b). All these excited states (¹G₄, ¹D₂, ³H₄) exhibit both rising and decay components in a profile on a microsecond time scale (FIG. 1c). This profile renders each different batch of nanoparticles a unique optical fingerprint, featured by a rather sophisticated multi-component lifetime behaviour.

TABLE 1
Summary of composition and size of 1→42 batches of UCNPs according to the five strategies*

| Sample | Tuning | | Migration | Sensitization | Inert | Size | Sample | τ²- |
|---|---|---|---|---|---|---|---|---|
| series | strategy | Core | Layer | Layer | Layer | (nm) | name | Dots |
| Yb—Tm | Tm³⁺ in core | 20Yb³⁺, 0.2Tm³⁺ | | | Y³⁺ | 45 | 1 | τ²-9 |
| | (20Yb³⁺) | 20Yb³⁺, 0.5Tm³⁺ | | | Y³⁺ | 44 | 2 | τ²-8 |
| | | 20Yb³⁺, 1Tm³⁺ | | | Y³⁺ | 42 | 3 | τ²-7 |
| | | 20Yb³⁺, 1.5Tm³⁺ | | | Y³⁺ | 42 | 4 | τ²-4 |
| | | 20Yb³⁺, 2Tm³⁺ | | | Y³⁺ | 43 | 5 | τ²-6 |
| | | 20Yb³⁺, 3Tm³⁺ | | | Y³⁺ | 42 | 6 | τ²-3 |
| | Tm³⁺ in core | 40Yb³⁺, 0.2Tm³⁺ | | | Y³⁺ | 51 | 7 | |
| | (40Yb³⁺) | 40Yb³⁺, 0.5Tm³⁺ | | | Y³⁺ | 51 | 8 | |
| | | 40Yb³⁺, 0.8Tm³⁺ | | | Y³⁺ | 53 | 9 | |
| | | 40Yb³⁺, 1Tm³⁺ | | | Y³⁺ | 53 | 10 | τ²-5 |
| | | 40Yb³⁺, 2Tm³⁺ | | | Y³⁺ | 53 | 11 | |
| | | 40Yb³⁺, 4Tm³⁺ | | | Y³⁺ | 54 | 12 | τ²-2 |
| | | 40Yb³⁺, 6Tm³⁺ | | | Y³⁺ | 51 | 13 | |
| | | 40Yb³⁺, 8Tm³⁺ | | | Y³⁺ | 53 | 14 | τ²-1 |
| Nd—Yb—Tm | Core only | 18Yb³⁺, 0.5Nd³⁺, 0.2Tm³⁺ | | | | 23 | 15 | |
| | Yb³⁺ in | 18Yb³⁺, 0.5Nd³⁺, 0.2Tm³⁺ | | 0Yb³⁺, 20Nd³⁺ | | 36 | 16 | |
| | sensitization | 18Yb³⁺, 0.5Nd³⁺, 0.2Tm³⁺ | | 5Yb³⁺, 20Nd³⁺ | | 36 | 17 | |
| | layer | 18Yb³⁺, 0.5Nd³⁺, 0.2Tm³⁺ | | 15Yb³⁺, 20Nd³⁺ | | 35 | 18 | |
| | | 18Yb³⁺, 0.5Nd³⁺, 0.2Tm³⁺ | | 20Yb³⁺, 20Nd³⁺ | | 35 | 19 | |
| | | 18Yb³⁺, 0.5Nd³⁺, 0.2Tm³⁺ | | 30Yb³⁺, 20Nd³⁺ | | 35 | 20 | |
| | | 18Yb³⁺, 0.5Nd³⁺, 0.2Tm³⁺ | | 45Yb³⁺, 20Nd³⁺ | | 35 | 21 | |
| | Inert layer | 18Yb³⁺, 0.5Nd³⁺, 0.2Tm³⁺ | | 0Yb³⁺, 20Nd³⁺ | Y³⁺ | 46 | 22 | |
| | | 18Yb³⁺, 0.5Nd³⁺, 0.2Tm³⁺ | | 5Yb³⁺, 20Nd³⁺ | Y³⁺ | 47 | 23 | |
| | | 18Yb³⁺, 0.5Nd³⁺, 0.2Tm³⁺ | | 15Yb³⁺, 20Nd³⁺ | Y³⁺ | 47 | 24 | |
| | | 18Yb³⁺, 0.5Nd³⁺, 0.2Tm³⁺ | | 30Yb³⁺, 20Nd³⁺ | Y³⁺ | 47 | 25 | |
| | | 18Yb³⁺, 0.5Nd³⁺, 0.2Tm³⁺ | | 45Yb³⁺, 20Nd³⁺ | Y³⁺ | 46 | 26 | |
| Nd—Yb—Er | Yb³⁺ in | 20Yb³⁺, 2Er³⁺ | 5Yb³⁺ | 15Yb³⁺, 20Nd³⁺ | | 49 | 27 | |
| | core/sensitization | 20Yb³⁺, 2Er³⁺ | 5Yb³⁺ | 30Yb³⁺, 20Nd³⁺ | | 50 | 28 | |
| | layer | 30Yb³⁺, 2Er³⁺ | 5Yb³⁺ | 30Yb³⁺, 20Nd³⁺ | | 49 | 29 | |
| | | 40Yb³⁺, 2Er³⁺ | 5Yb³⁺ | 15Yb³⁺, 20Nd³⁺ | | 49 | 30 | |
| | Er³⁺ in core | 30Yb³⁺, 2Er³⁺ | 5Yb³⁺ | 15Yb³⁺, 20Nd³⁺ | | 50 | 31 | |
| | | 30Yb³⁺, 8Er³⁺ | 5Yb³⁺ | 15Yb³⁺, 20Nd³⁺ | | 49 | 32 | |
| | Er³⁺/Yb³⁺ in core | 20Yb³⁺, 1Er³⁺ | 5Yb³⁺ | 15Yb³⁺, 20Nd³⁺ | Y³⁺ | 59 | 33 | |
| | | 20Yb³⁺, 1.5Er³⁺ | 5Yb³⁺ | 15Yb³⁺, 20Nd³⁺ | Y³⁺ | 60 | 34 | τ²-11 |
| | | 30Yb³⁺, 8Er³⁺ | 5Yb³⁺ | 15Yb³⁺, 20Nd³⁺ | Y³⁺ | 59 | 35 | τ²-10 |
| | Thickness of | 20Yb³⁺, 2Er³⁺ (22 nm) | 5Yb³⁺ | 15Yb³⁺, 20Nd³⁺ | Y³⁺ | 48 | 36 | |
| | sensitization | 20Yb³⁺, 2Er³⁺ (30 nm) | 5Yb³⁺ | 15Yb³⁺, 20Nd³⁺ (6 nm) | Y³⁺ | 59 | 37 | |
| | layer and core | 20Yb³⁺, 2Er³⁺ | 5Yb³⁺ | 15Yb³⁺, 20Nd³⁺ (10 nm) | Y³⁺ | 66 | 38 | τ²-12 |
| | Yb³⁺ in | 20Yb³⁺, 2Er³⁺ | 5Yb³⁺ | 5Yb³⁺, 20Nd³⁺ | Y³⁺ | 61 | 39 | τ²-14 |
| | core/sensitization | 20Yb³⁺, 2Er³⁺ | 5Yb³⁺ | 30Yb³⁺, 20Nd³⁺ | Y³⁺ | 60 | 40 | |
| | layer | 30Yb³⁺, 2Er³⁺ | 5Yb³⁺ | 5Yb³⁺, 20Nd³⁺ | Y³⁺ | 61 | 41 | τ²-13 |
| | | 30Yb³⁺, 2Er³⁺ | 5Yb³⁺ | 15Yb³⁺, 20Nd³⁺ | Y³⁺ | 60 | 42 | |

*concentrations given in the table are in mol %

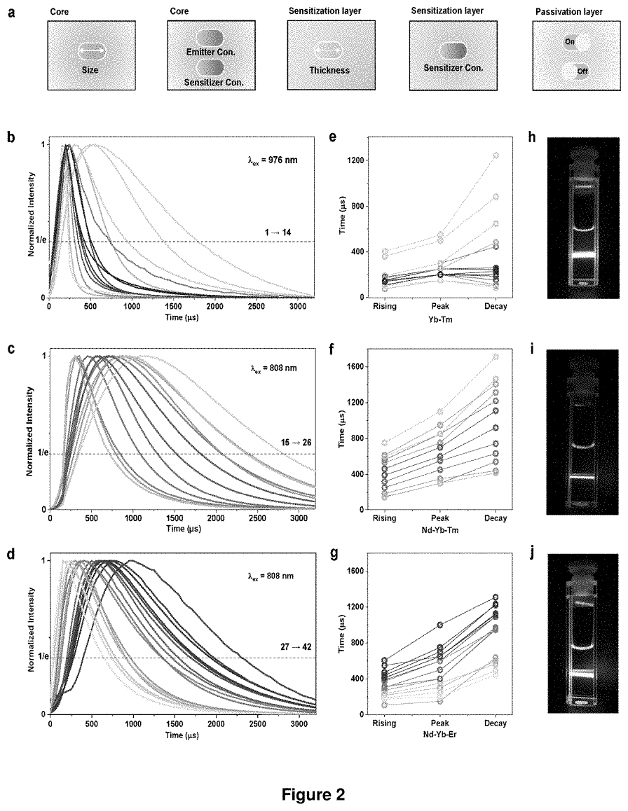

As illustrated in FIG. 2a, the strategies include the tuning of the core size, doping concentrations of emitters and sensitizer Yb³⁺ in the core, the thickness of the core/sensitization layer, and the doping concentration of Yb³⁺ in the sensitization layer, as well as the adding of a passivation inert layer.

It will be appreciated that in preparing a library, one or more of these strategies may be adopted. In some embodiments, all five strategies are adopted. Using all five strategies (see Table 1), fourteen (1→14 in FIG. 2b), twelve (15→26 in FIG. 2c), and sixteen (27→42 in FIG. 2d) batches of three series of τ²-Dots were synthesized which show finely tunable τ² profiles under NIR excitation at 976 nm or 808 nm. Though samples from the same doping series exhibit very similar emission colours, i.e., blue for the Yb—Tm series (FIG. 2h), violetish blue for Nd—Yb—Tm series (FIG. 2i), yellowish-green for the Nd—Yb—Er series (FIG. 2j), their lifetime profiles display very differently in the time domain. Values in FIG. 2e-2g further quantitatively map the large dynamic ranges of rising time, peak moment, and decay time distributions in identifying each batch of τ²-Dots samples.

Preferably the nanoparticles in step (a) are selected from: core-multi-shell β-NaYF₄: Nd³⁺, Yb³⁺, Tm³⁺ UCNPs, core-multi-shell β-NaYF₄: Nd³⁺, Yb³⁺, Er³⁺ UCNPs and core-shell β-NaYF₄: Yb³⁺, Tm³⁺ UCNPs. In some embodiments, the nanoparticles in step (a) are core-multi-shell β-NaYF₄: Nd³⁺, Yb³⁺, Tm³⁺ UCNPs, core-multi-shell β-NaYF₄: Nd³⁺, Yb³⁺, Er³⁺ UCNPs and core-shell β-NaYF₄: Yb³⁺, Tm³⁺ UCNPs, such that the library is based on three UCNP types as shown in Table 1. However, those skilled in the art will appreciate that the library may be based on other UCNPs, and indeed nanoparticles more generally, as long as their optical uniformity and tunability of optical fingerprints, e.g. in the spectrum, meet the requirement discussed herein.

### Optical Uniformity of Single τ²-Dots

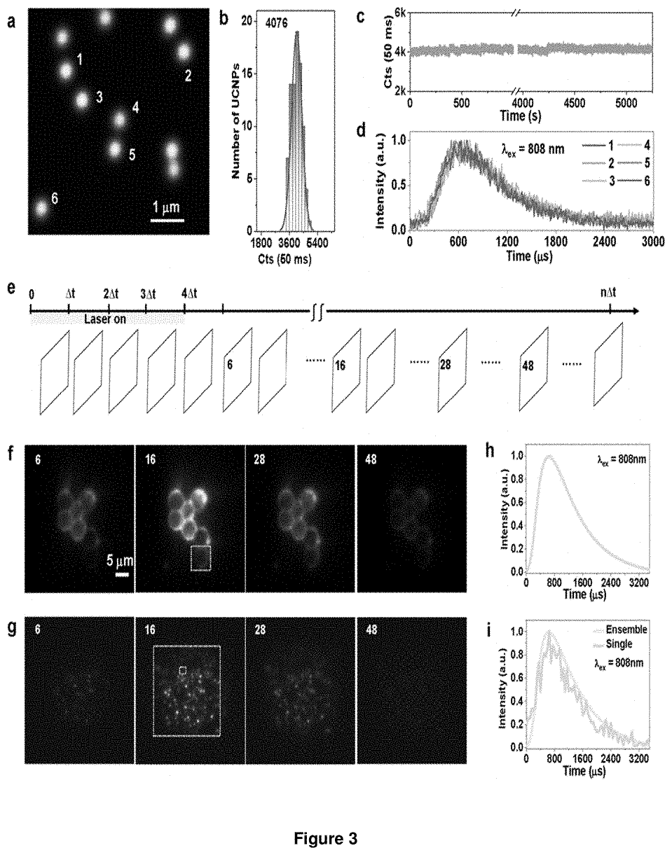

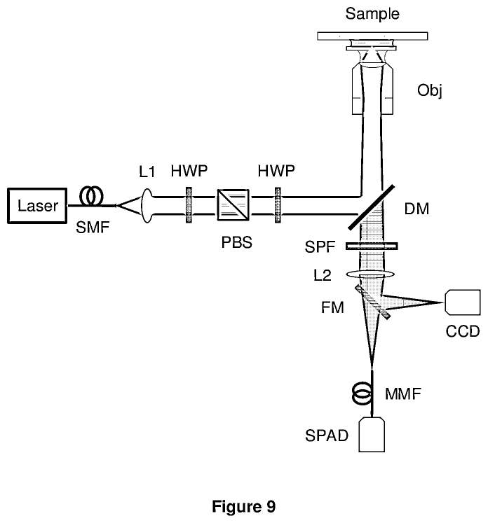

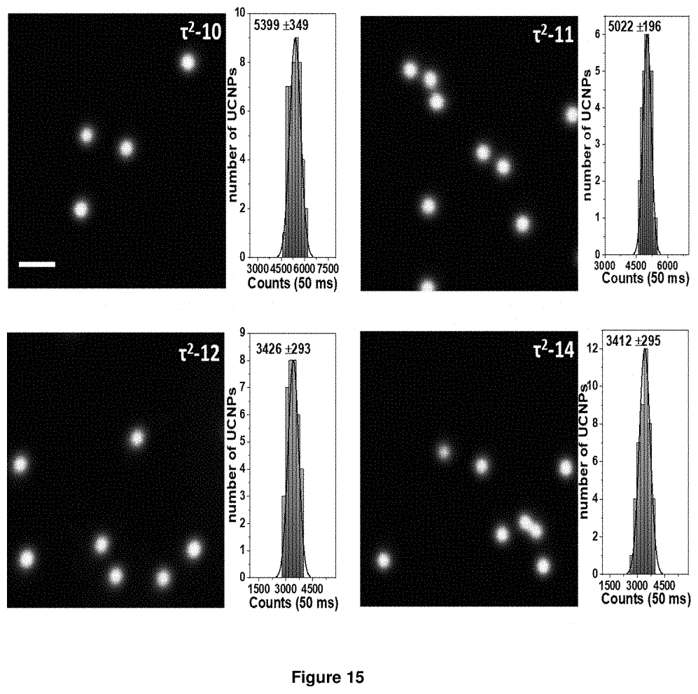

The applicant has found that despite the large dynamic ranges of lifetime profiles that can be encoded in different batches of τ²-Dots, the difference between each encoded optical fingerprint can be hidden at the ensemble level. Therefore, the single nanoparticle spectroscopy method should be adopted to verify the optical uniformity of single τ²-Dots. Here, fourteen batches of τ²-Dots (namely τ²-1 to τ²-14 in Table 1) were selected in the Yb—Tm series (τ²-1 to τ²-9) and Nd—Yb—Er series (τ²-10 to τ²-14) to perform the decoding experiment at single nanoparticle level. Using a confocal microscopy setup (FIG. 9), the single nanoparticle optical characterization result (FIGS. 3a and 3b) shows high degrees of brightness (e.g., 81,520 photon counts per second for τ²-13), optical uniformity (CV of 8.1%) (see other 4 batches of Nd—Yb—Er τ²-Dots in FIG. 15), and stability of single τ²-Dots (FIG. 3c), ideal for long-term imaging and decoding of the optical fingerprint. As shown in FIG. 3d, the unique and detectable fingerprint has been successfully assigned to every single τ²-Dot. More impressively, the characteristic lifetime fingerprints of single dots, as long as from the same batch of synthesis, are consistently uniform.

### Wide-Field Time-Resolved Microscopy

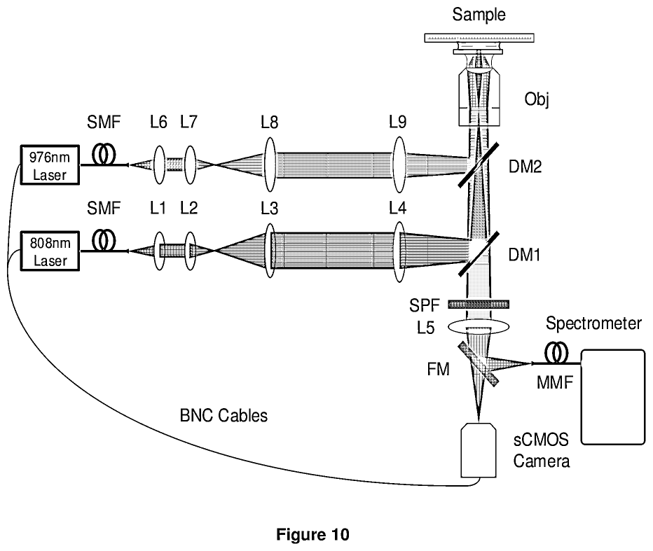

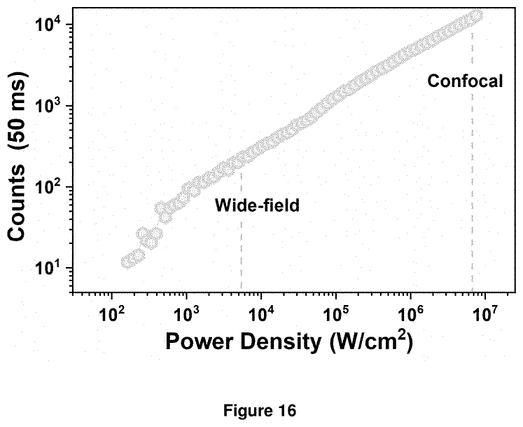

Confocal scanning microscopy allows illumination power up to 106 W/cm² to excite every single nanoparticle by scanning across each pixel, but of which the scanning mode dramatically limits the throughput in the decoding process. A wide-field microscope was therefore developed with an intensifier coupled CMOS camera for time-resolved imaging (FIG. 10). Under the wide-field microscopy, moderate continuous-wave excitation power density (5.46 kW/cm²) sacrifices the brightness of each τ²-Dot by nearly two orders of magnitude (FIG. 16), but the wide-field microscopy enhances the decoding throughput by orders of magnitude, compared with the point scanning confocal setup. As shown in FIG. 3e, the sequence of time-resolved imaging consists of 75 frames (n=75), each recording the time-gated window period (Δt) of 50 μs.

### Nanoscale Optical Multiplexing of Single τ2-Dots

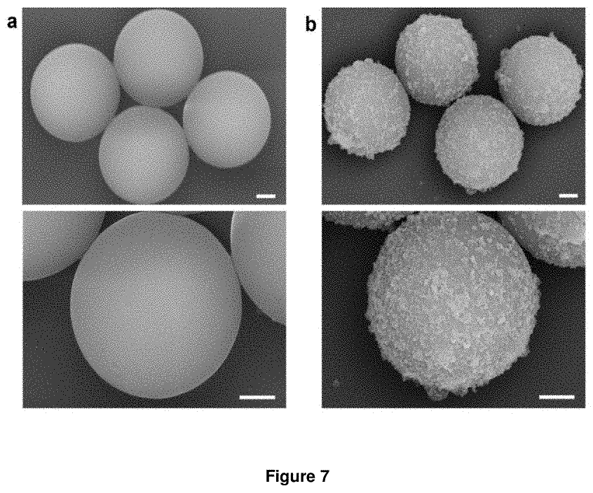

Compared to the conventional micron-sized beads, optical codes created on nanoscopic-sized τ²-Dots can significantly increase the capacity of coding information, which takes optical super capacity multiplexing into the region smaller than the optical diffraction limit. To illustrate this opportunity and challenge, 5 μm polystyrene beads were stained with τ²-13 dots (FIG. 7) and their time-resolved upconversion images collected under a wide-field microscope. Within an illumination area of 28 μm in diameter, a typical image only contains less than ten micron-sized beads (FIG. 3f), while in contrast, there are hundreds of single τ²-13 dots within the same area (FIG. 3g). Each single micron bead shows a smooth τ² profile (FIG. 3h), but the curve from a single τ² dot (FIG. 3i) has some significant level of noise, due to the limited amount of detectable signal within each 50 μs time-gated window.

### Extraction of High Dimensional Fingerprints

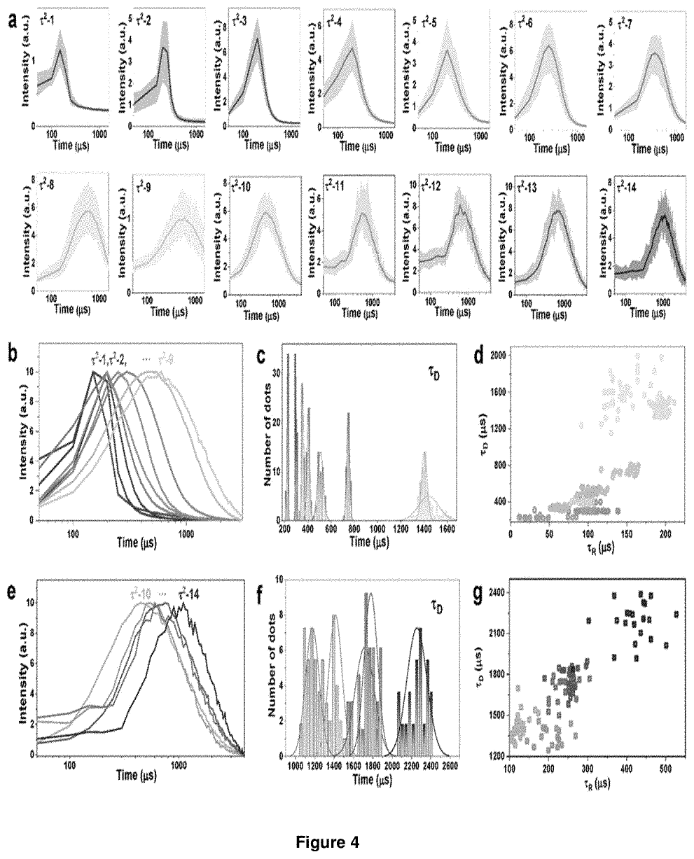

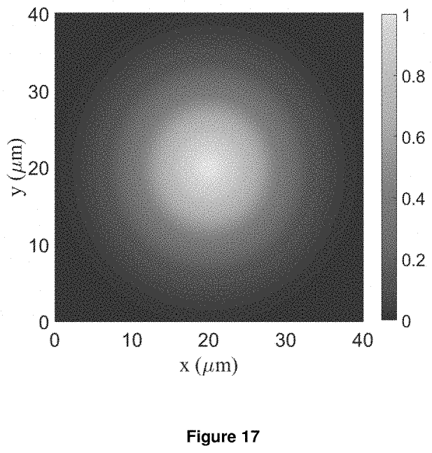

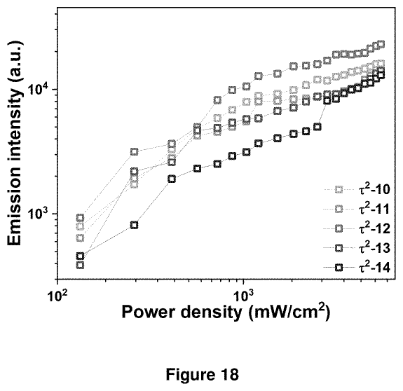

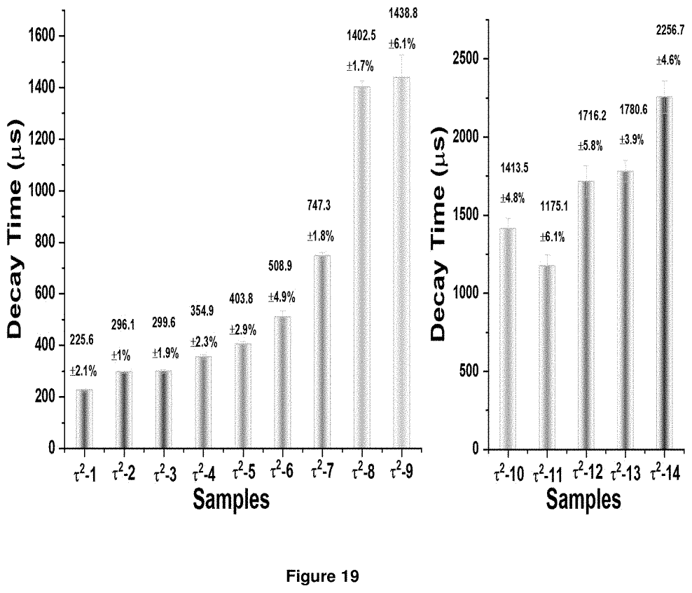

Using the wide-field time-resolved microscope, the lifetime curves of more than 20 single τ²-Dots from each batch were measured and their lifetime profiles are presented in FIG. 4a. Although some detectable variations of the lifetime curves from dot to dot, caused by the illumination distribution (FIG. 17) and power-dependent intensities (FIG. 18), distinctive characteristics of each τ²-Dot and their lifetime tunability over a large dynamic range are clear (FIGS. 4b and 4e). Through the distribution statistics (FIGS. 4c and 4f), it was found that most of the τ²-Dots have their m values distributed uniformly with a small CV (<10%, FIG. 19) and a small degree of overlap between each population, which is favourable for the decoding process. Four pairs of τ²-Dot populations, including τ²-2 vs τ²-3, τ²-4 vs τ²-5, τ²-8 vs τ²-9, and τ²-13 vs τ²-14, show significant overlap. Strikingly, by adding one more indicator, extracted from the lifetime fingerprint profile, i.e., τ_{R}, the two pairs of populations (τ²-4 vs τ²-5, τ²-8 vs τ²-9) could be well distinguished (FIG. 4d).

### Deep Learning Approach

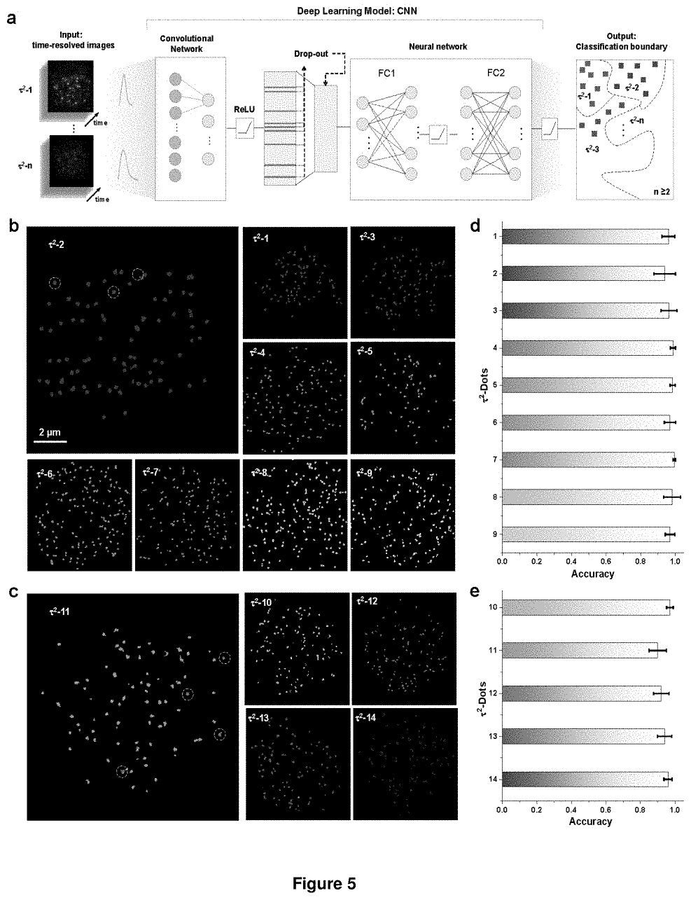

Deep learning is an emerging technique showing strong ability to classify highly non-linear datasets. Here an opportunity was offered by both the controlled growth of highly optically uniform single nanoparticles and subsequent image analysis to obtain lifetime fingerprints of single dots, which can generate a large set of high-quality data to train the machine in deep learning. By collecting the sequences of time-dependent frames of images, we extracted the values of the normalized τ² profiles at 75 time moments between 0-3750 μs as the data source of input for training, in which we first pre-process the as-collected images by only selecting the imaging data from single nanoparticles. As shown in FIG. 5a, we employed a convolutional network and a fully connected network with two layers (FC1 and FC2) to define the feature coverage for each batch of τ²-Dots (the classification boundaries).

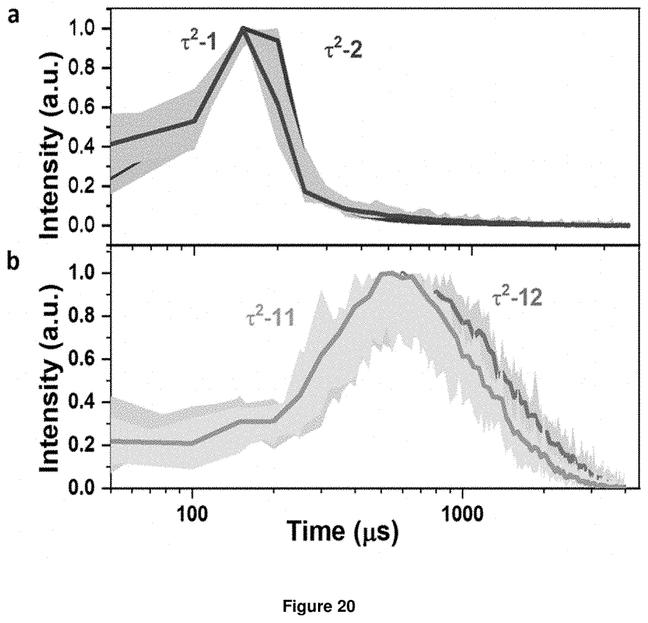

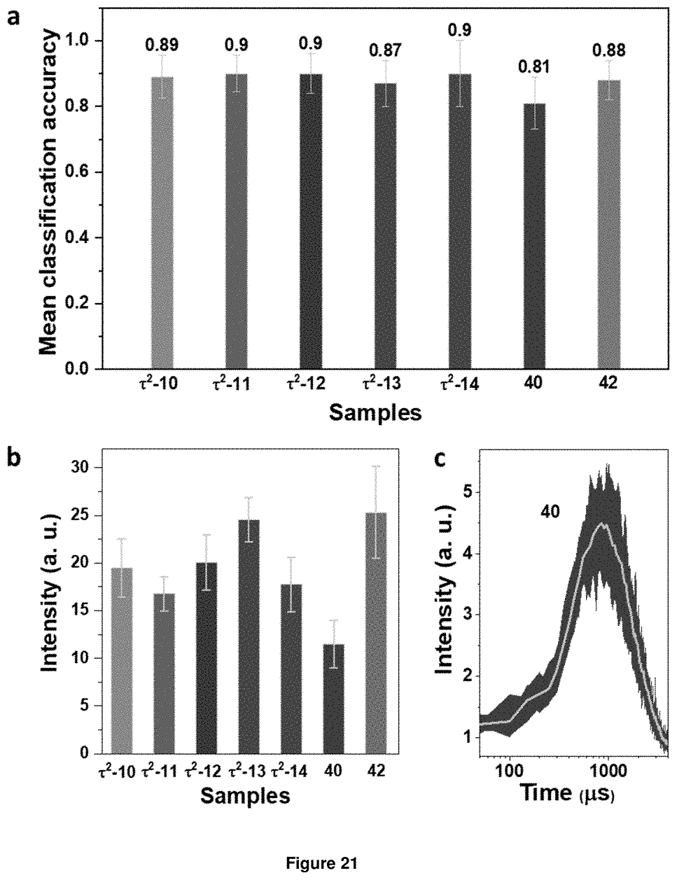

We train the machine by the database of 14 batches of τ²-Dots with two series independently (τ²-1 to 9 and τ²-10 to 14) and challenge the established neural networks to recognize every single τ²-Dot. To do this, we first collected seven sets of time-resolved sequences of images from each type of τ²-Dots sample, and each image data contains the lifetime fingerprints of 50 to 200 single nanoparticles after data preselection of single τ²-Dots. We use any six sets of imaging data from each type of τ²-Dots to train the machine first to establish a neural network, and use the last set of data as validation analytes. A typical set of visualized result for each τ²-Dot sample was displayed in FIGS. 5b and 5c. A small amount of mottled dots (e.g., in images of τ²-2 and τ²-11) represent the error recognition, which is mainly caused by the samples with similar lifetime curve features (FIG. 20). We then run the experiment of training and validation for another 50 times, each time randomly chose one set of data as the validation target and the other six sets to train the neural networks, which resulted in the statistical distributions of classification accuracy with error bars, displayed in FIGS. 5d and 5e. We achieved the mean classification accuracies for each τ²-Dot sample, with all the values approaching the unity. The capacity of nanoscale multiplexing can be significantly determined by the brightness of single nanoparticles and the noise background, which explains the relatively broad distributions of τ² profiles for the batches of τ²-Dot samples with relatively low brightness, and therefore less accurate recognition results can be achieved by the machine intelligence (FIG. 21). Nevertheless, this experiment confirms the great potentials for the lifetime profiles of each τ²-Dot to be used for nanoscale super-capacity optical multiplexing, assisted by deep learning.

### Potential Applications of τ²-Dots

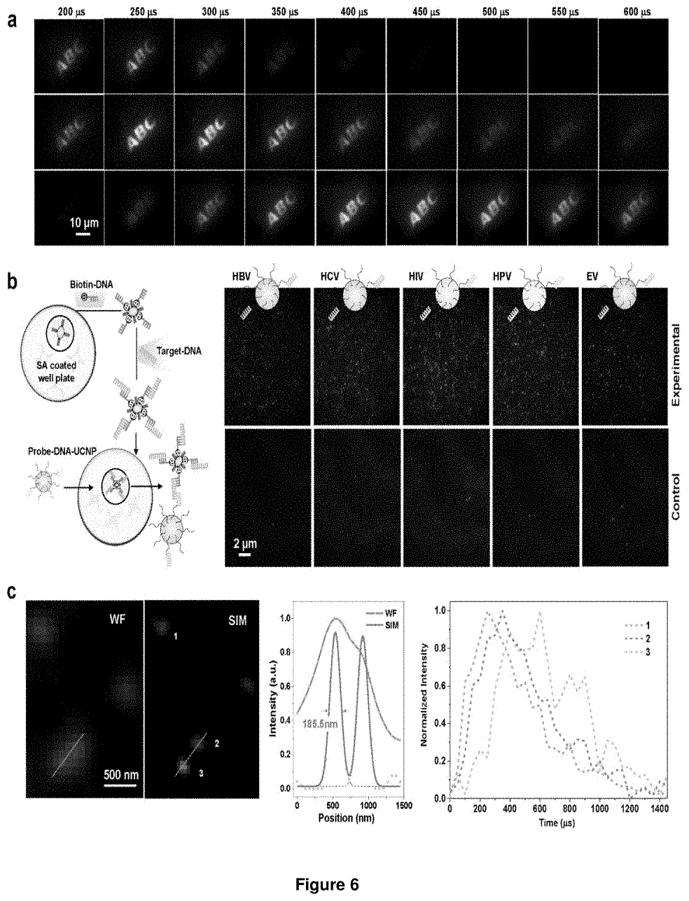

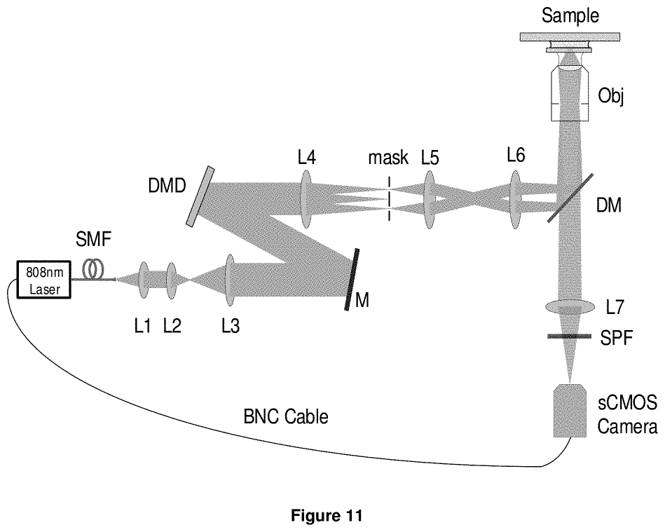

The nanoscale super-capacity optical multiplexing opens a new horizon for many applications. Using the time-domain τ²-profiles, different batches of materials emitting the same colour can be used to develop the new generation of dynamic anti-counterfeiting security inks, as illustrated in FIG. 6a. Another unparalleled potential is to use nanoscale super-capacity multiplexing for high-throughput single molecular assay, which is superior to conventional suspension array assays based on microspheres. As a result of a proof of the principle experiment, in FIG. 6b, we designed and functionalized the five kinds of τ²-Dots to simultaneously detect the five species of pathogenetic DNA sequences (see Table 2)—hepatitis B virus (HBV), hepatitis C virus (HCV), human immunodeficiency virus (HIV), human papillomavirus type-16 (HPV-16), and Ebola virus (EV). Through a wide-field microscope, and compared with the control groups, we concluded that each τ²-Dots were highly specific. Moreover, as shown in FIG. 5c, we demonstrate that the wide-field images of τ²-Dots with different lifetime profiles can be super-resolved using our latest development of upconversion structure-illumination microscopy (U-SIM) (FIG. 11) with a resolution of 185.5 nm.

TABLE 2
Five kinds of pathogenetic DNA sequences
conjugated with 5 batches of t2-Dots

| Virus | Capture DNA | Target DNA | Probe DNA |
|---|---|---|---|
| Hepatitis B virus | 5′-/5AmMC12/ | 5′-TTG GCT TTC | 5′-ACT GAA AGC |
| (HBV) | ATC ATC | AGT TAT | CAA |
| | CAT ATA-3′ | ATG GAT GAT-3′ | /iSpC12//3Bio/-3′ |
| hepatitis C virus | 5′-/5AmMC12/ | 5′-GGC GTT GAC | 5′-CCC GTC AAC |
| (HCV) | CGT GTA | GGG GTC | GCC |
| | AGT GAC-3′ | ACT TAC ACG-3′ | /iSpC12//3Bio/-3′ |
| Human | 5′-/5AmMC12/ | 5′-AGA AGA TAT | 5′-CAA ATA TCT |
| immunodeficiency | GTC ATG | TTG GAA | TCT |
| virus | TTA TTC-3′ | TAA CAT GAC-3′ | /iSpC12//3Bio/-3′ |
| (HIV) | | | |
| Human | 5′-/5AmMC12/ | 5′-ATT TGC TGC | 5′-TAT GCA GCA |
| papillomavirus | AAT GCT | ATA AGC | AAT |
| type-16 | AGT GCT-3′ | ACT AGC ATT-3′ | /iSpC12//3Bio/-3′ |
| (HPV-16) | | | |
| Ebola virus | 5′-/5AmMC12/ | 5′-GGA GTA AAT | 5′-AAC ATT TAC |
| (EV) | ATA CTG | GTT GGT | TCC |
| | TTC TCC-3′ | GAA CAG TAT-3′ | /iSpC12//3Bio/-3′ |

### Materials and Methods

### Synthesis of UCNPs

The NaYF₄ core nanoparticles were synthesized using a coprecipitation method¹. In a typical procedure, 1 mmol RECl₃ (RE=Y, Yb, Nd, Er, Tm) with different doped ratios together with 6 mL oleic acid and 15 mL 1-octadecene were added to a 50 ml three-neck round-bottom flask under vigorous stirring. The resulting mixture was heated at 150° C. for 40 mins to form lanthanide oleate complexes. The solution was cooled to 50° C., and 6 mL methanol solution containing 2.5 mmol NaOH and 4 mmol NH₄F was added with vigorous stirring for 30 mins. Then the mixture was slowly heated to 150° C. and kept for 30 mins under argon flow to remove methanol and residual water. Next, the solution was quickly heated at 300° C. under argon flow for 1.5 h before cooling down to room temperature. The resulting core nanoparticles were collected and redispersed in cyclohexane with 5 mg/mL concentration after washing with cyclohexane/ethanol/methanol several times. Three series of core nanoparticles were synthesized (NaYF₄: Yb, Tm; NaYF₄: Yb, Nd, Tm; NaYF₄: Yb, Er) with different doping concentrations using the same above method.

The precursors were prepared using the above procedure until the step where the reaction solution was slowly heated to 150° C. after adding NaOH/NH₄F solution and kept for 30 mins. Instead of further heating to 300° C. to trigger nanocrystal growth, the solution was cooled down to room temperature to yield the shell precursors.

The core-shell and core-multi-shell nanoparticles were prepared by a layer-by-layer epitaxial growth method. The pre-synthesized NaYF₄ core nanoparticles were used as seeds for shell modification. 0.2 mmol as-prepared core nanocrystals were added to a 50 ml flask containing 3 ml OA and 8 ml ODE. The mixture was heated to 150° C. under argon for 30 min, and then further heated to 300° C. Next, a certain amount of as-prepared shell precursors were injected into the reaction mixture and ripened at 300° C. for 2 mins, followed by the same injection and ripening cycles for several times to get different shell thickness. Finally, the slurry was cooled down to room temperature and the formed core-shell nanocrystals were purified according to the same procedure used for the core nanocrystals. The core-multi-shell nanoparticles were also prepared by the epitaxial growth method described above and the core-shell nanoparticles were used as the seeds.

Preparation of τ²-Dots (τ²-13) Tagged Microbeads

The polystyrene (PS) microbeads (d=5 μm; Sigma-Aldrich) solution was processed by swelling 5 μl (10% w/v) of PS beads with 137 μl of an 8% (v/v) chloroform solution in butanol. 40 μl (8 mg/ml) τ²-13 dots in cyclohexane was added to the above PS suspension. The solution was vortexed after adding the τ²-Dots. After incubating at 25° C. for 3 hours, the beads were washed four times, alternating between ethanol and cyclohexene. After washing, the τ²-Dots embedded beads were dispersed in ethanol and then one drop of the beads was air-dried on the surface of a coverslip for optical measurements.

### Material Characterizations

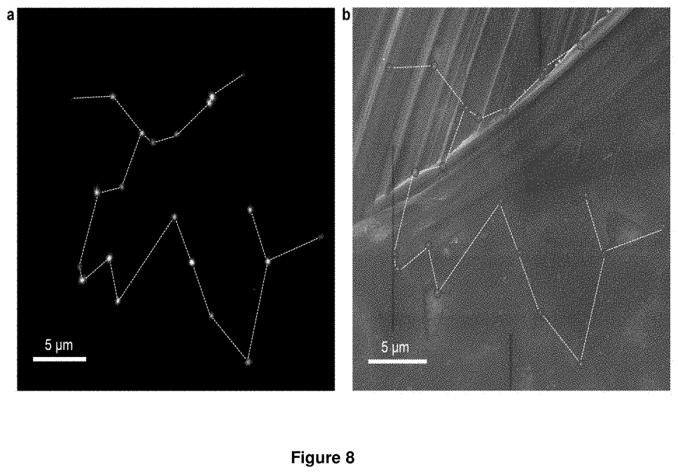

The morphology characterization of the nanoparticles was performed by transmission electron microscopes of JEOL TEM-1400 at an acceleration voltage of 120 kV and JEOL TEM-2200FS with the 200 kV voltage. The cyclohexane dispersed UCNPs were imaged by dropping them onto carbon-coated copper grids. The surface morphology characterization of the PS beads (see FIG. 7) and the light-electron microscopic correlation experiment (see FIG. 8) were performed by using a Zeiss Supra 55VP Scanning Electron Microscope (SEM) operated at 20.00 kV.

### Preparation of Sample Slides

To prepare a sample slide for single nanoparticle measurement, a coverslip was washed with pure ethanol by ultrasonication, followed by air-drying. 20 μl of the τ²-dots (0.01 mg/ml) in cyclohexane was dropped onto the surface of a coverslip. After being air-dried, the coverslip was put over a clean glass slide and any air bubbles were squeezed out by gentle force before measurement.

### Confocal Imaging and Lifetime Measurement

A stage-scan confocal microscope was built for the intensity and lifetime measurements of single τ²-Dots, as shown in FIG. 9. The excitation source of 808 nm single-mode polarized laser was focused onto the sample through a 100× objective lens (UPLanSApo100×, oil immersion, NA=1.40, Olympus Inc., JPN). The emission from the sample was collected by the same objective lens and refocused into an optical fibre which has a core size matching with the system first Airy disk. The fluorescence signals were filtered from the laser by a short-pass dichroic mirror (DM, ZT785spxxr-UF1, Chroma Inc., USA) and a short pass filter (SPF, ET750sp-2p8, Chroma Inc., USA). A single-photon counting avalanche photodiode (APD, SPCM-AQR-14-FC, Excelitas Inc., USA) was connected to the multi-mode fibre (MMF, M42L02, Thorlabs Inc., USA) to detect the emission intensity. The scanning was achieved by moving the 3D piezo stage. Every single nanoparticle showed a Gaussian spot in the confocal scanning microscopic image. The maximum brightness value (photon counts) of each Gaussian spot was used to represent the brightness of that single nanoparticle. More than 20 single nanoparticles were evaluated to calculate the mean brightness.

For the lifetime measurement, the diode laser was modulated to produce 200 μs excitation pulses. The photon-counting SPAD was continuously switched on to capture the long-lifetime luminescence. For each time point, the gate-width is 5 μs with an accumulation of 10000 times. The pulsed excitation, time-gated data collection and the confocal scanning were controlled and synchronized using a multifunction data acquisition device (USB-6343, National Instruments) and a purpose-built LabVIEW program.

### Wide-Field Spectrum and Lifetime Measurement

A wide-field fluorescence microscope was built, as shown in FIG. 10, to acquire the fluorescence lifetime image sequences of τ²-Dots. A single-mode diode-pumped solid-state laser (LU0808M250, Lumics Inc., GER, 808 nm, the excitation power density of 5.46 kW/cm²) was used to excite the τ²-Dots after expanding the laser beam by three times. The emission of τ²-Dots was collected by a high NA objective lens (UPLanSApo100×, oil immersion, NA=1.40, Olympus Inc., JPN) and separated from the laser reflection by a short-pass dichroic mirror (DM1, ZT785spxxr-UF1, Chroma Inc., USA) and a short pass filter (SPF, ET750sp-2p8, Chroma Inc., USA), then focused by a tube lens to the time-resolved sCMOS camera (iStar sCMOS, Andor Inc., UK). The camera also functions as the pulse modulator of an exciting laser beam via a BNC cable. By applying the Kinetics Mode of the camera and Integrate-On-Chip (IOC) at 250 Hz, the lifetime image sequences of 75 frames were acquired from 0 μs to 3750 μs with a time gate of 50 μs, under the laser excitation pulse of 0-200 μs. The IOC mode enabled the accumulation of fluorescence signal with the greatly improved signal-to-noise ratio. To measure the fluorescence lifetime image sequences of τ²-Dots under 976 nm excitation, a single-mode 976 nm laser (BL976-PAG900, Thorlabs Inc., USA, the excitation power density of 8.7 kW/cm²) was added in the setup as the excitation light. After collimation, the excitation beam was expanded by 2.5 times and then reflected by the short-pass dichroic mirror (DM2, T875spxrxt-UF1, Chroma Inc., USA), and focused through the objective lens to the sample slide. The fluorescence signals can also be coupled into a multi-mode fibre (MMF, M24L02, Thorlabs Inc., USA) by switching a flip mirror and then detected by a miniature monochromator (iHR550, Horiba Inc., JPN) for measuring upconversion emission spectra. The spectral region ranged from 400 to 750 nm.

### Data Processing and Networks for Deep Learning

To perform single nanoparticle-based machine learning, data processing was performed to select the single nanoparticles in the collected images. The brightest frame (maximum mean brightness) from the 75 frame images was selected. Then the peak pixel of each bright spot was found. For each peak, a 40-pixel by 40-pixel region of interest (ROI) was cropped centred on the peak. In each ROI, the image was segmented with the OTSU threshold and get a binary mask. Considering that two adjacent peaks might be connected in the binary mask, watershed segmentation was employed on the binary mask to get the boundaries of each peak. Finally, all the spots were sorted by their peak intensity and divided all spots into four groups (Q1 to Q4) according to their peak intensities. The Q1-Q4 represented 4 intensity thresholds to classify the groups. The spots were counted as the single nanoparticles when the peak intensities within the statistical range of single particle intensity (eg. 8000±1000 for τ²-13, equaling to Q2 group). After filtering out all the aggregated spots, an image that only involves single nanoparticles was obtained. After that, the image sequence was transformed into multiple single nanoparticle sequences. For example, if 100 particles were identified as single nanoparticles in an image sequence, this image sequence was decomposed into 100 particle sequences.

The artificial neural networks (ANN) were implemented in python using the PyTorch package (https://pytorch.org/). We extracted the normalized time-domain fluorescence intensity sequences of single nanoparticles as the input for deep learning. We performed the aforementioned data processing for all the 14 τ²-Dots. About 500 single nanoparticles were randomly selected for each τ²-Dots as the training sets, where their lifetime features and types were known. ˜100 single nanoparticles were used as the validation sets. There were five key aspects during determining the networking architecture: 1) the number of layers in the convolutional network; 2) the number of filters in each 1D convolutional layer; 3) whether to use activation function; 4) the number of neurons in each fully connected (FC) layer; 5) the keep probability for the dropout regularization scheme. We started with the network structure of one convolutional layer with 10 filters and two fully connected layers with 10 neurons for each of them.

The number of neurons was first determined in each fully connected layer ranging from 10 to 1000. Given one convolutional layer with 10 filters, the network obtained satisfactory results when the number of neurons in each FC layer was around 500. Given the above two FC layers, we started to determine the number of convolutional layers and the number of filters for each layer. The network obtained satisfactory results when using two convolutional layers with 50 filters in the first layer and 20 filters in the second layer. Then, given the above convolutional layers, we further adjusted the number of neurons for each FC layer, and found 100-200 neurons in each layer can obtain satisfactory results. With the above conductions, the network structure was temporarily determined as two convolutional layers with 50 filters in the first layer and 20 filters in the second layer followed by two FC layers with 100 neurons for each layer. With this network structure, we validated the network performance when activation functions or/and dropout scheme was/were introduced. Three activation functions have been validated during this procedure, which were ReLU, ReLU6 and RReLU. The keep probability of the dropout scheme was determined in the range from 0.5 to 0.9. After the above adjustment of the network, we went back to adjust the number of neurons in FC layers and obtained the final network architecture as below.

The fingerprint retrieval network contained two convolutional networks and two fully connected networks. The two 1D convolutional layers used the element-wise function ReLU6(x)=min(max(0,x), 6). There were 50 filters in the first 1D convolutional layer using a kernel of size 3 and the stride size was 2. The second 1D convolutional layer has 20 filters with a kernel of size 2 and the stride size was 1. The two fully connected networks contained two layers with 150 (FC1) neurons in the first layer and 100 (FC1) neurons in the second layer, and the element-wise function was also employed for each layer. We applied a dropout regularization scheme with 80% keep probability for the fully connected part. During training, the output layer neuron whose index corresponds to the input binary number was set to “1” while the other neuron activations were kept at “0”. A variant of the stochastic gradient descent (SGD) algorithm (“Adam”) was applied to train the parameters in the network through a randomly shuffled batch of size 200. We used the categorical cross-entropy loss, a learning rate of 0.005 and train the network for 50 epochs.

The classification effectiveness of convolutional neural networks was evaluated by the mean and deviation of the classification accuracy of 50 randomly sampled experiments. We have 7 sets of image sequences of each sample and run 50 experiments of training-and-testing to compute the average error and deviation. In each experiment, we randomly selected one set of image sequences for test particles. For 14 batches of nanoparticles we selected 14 image sequences. The data of single nanoparticles in the rest image sequences were used as the training set in the training algorithm section, where their lifetime features were available, but the label was unknown until computing the model error. After one training-and-testing process, the testing error for 14 image sequences was obtained. The mean and deviation of errors were computed through 50 random selections.

### Anti-Counterfeiting Experiment

The time-domain anti-counterfeiting by using three types of τ²-Dots was based on the spatial modulation of the excited patterns on the sample plane. A digital micro-mirror device (DMD) was added in the wide-field optical system as the spatial light modulator to generate excitation patterns of the ABC alphabet. The laser beam illuminated the DMD after beam collimation and expansion. Then the illuminated alphabet patterns were imaged on the sample plane

### DNA Assay Experiment

Post-synthesis surface modification was adopted to transfer the τ²-Dots into hydrophilic and biocompatible before bioconjugation with DNA oligonucleotides. Surface modification was performed via ligand exchange with a block copolymer composed of hydrophilic block poly(ethylene glycol) methyl ether acrylate phosphate methacrylate (POEGMA-b-PMAEP)². In a typical procedure, 500 μl of OA-coated τ²-Dots (20 mg/mL) were dispersed in tetrahydrofuran (THF). Then the OA-capped τ²-Dots in THF were mixed with 5 mg copolymer ending in carboxyl group in 2 mL THF. The above mixture was sonicated for one min followed by incubation in a shaker overnight at room temperature. The polymer-coated τ²-Dots were purified four times by washing/centrifugation at 14860 rpm for 20 min with water to obtain carboxyl group modified τ²-Dots. The supernatant was removed and the nanoparticles were dispersed in water for further conjugation with DNA.

We selected five couples of pathogen-related genetic sequences in the short length of 24 bases (HBV, HCV, HIV, HPV-16, EV). The protocol of carbodiimide chemistry was adopted to conjugate the carboxyl group on the polymer with the amine groups of probe DNA molecules. The five groups of carboxyl-τ2-Dots were re-activated by the EDC (100-folder molar ratio to carboxyl-τ2-Dots) in HEPES buffer (0.2 mM, pH 7.2) with slightly shaking at room temperature for 30 mins. The five groups of NH₂-DNA (100 uM) was added into the above solution with 600 rpm shaking for the reaction of 3 h, respectively. The activated carboxyl-τ2-Dots were washed/centrifuged at 14680 rpm cycle two times to remove EDC and resuspended in HEPES buffer to obtain probe DNA-polymer-τ2-Dots.

The Streptavidin with a concentration of about 0.5 μg/mL in 200 μL PBS buffer was coated on the 5 pairs of 96-well plates and incubated 4 h at room temperature. Following by removal of the supernatant, 200 pmol biotinylated-capture DNA in 200 μL PBS was added into the well and incubated overnight at 4° C. for further immobilization. Washing the plates 3 times with PBS buffer after the reaction, then 200 μL of blocking with 1% casein buffer was added to each well and incubated at room temperature for 1 h. The Target-DNA in 200 uL Tris buffer was added to five of the experimental wells and incubated at room temperature for 2 h, while the five corresponding control wells were added Tris buffer without Target-DNA. After washing 3 times with Tris buffer, 100 μL complementary DNA-functionalized τ²-Dots in reaction buffer contains 0.1% casein and 5 mM NaF in Tris were added to react 1 h. Then washing the wells 3 times and the well was ultimately dissolved in 100 μl Tris-5 mM NaF before detecting the images.

### SIM Imaging Experiment

Structured illumination microscopy (SIM), as a wide-field super-resolution technique, was based on the spatial modulation of the excited patterns on the sample plane. In this work, a digital micro-mirror device (DMD, DLP 4100, Texas Instruments Inc., USA) was used as the spatial light modulator to generate excitation patterns. DMD contained an array of 1024×768 micro-mirrors on the chip. The size of each micromirror was 13.68×13.68 pmt. For each of the micro-mirrors, the physical size was slightly less than 13.68 μm due to the fill factor of 91%. Each micro-mirror can be tilted to two positions along its diagonal: ±12° tilt to deflect the incident light beam away from the optical path. These micro-mirrors can be controlled independently to modulate the amplitude of incoming light to generate arbitrary illumination patterns.

As shown in FIG. 11, the optical system for the time-resolved SIM was built based on conventional widefield fluorescence microscopy (FIG. 10) with proper modification. In the reconstruction of super-resolution image series, nine raw image series were acquired with nine illuminating patterns, corresponding to three different angular orientations (θ1=0°, θ2=60° and θ3=120°) and three different phase shifts (φ1=0°, φ2=120° and φ3=240°). Then all nine frequency spectra, for each frame of these series were obtained by applying a Fast Fourier Transform algorithm to these raw images. After separation of the spectrum, all nine frequency components were shifted to their true positions to reconstruct the final SIM images. All the data was reconstructed using ImageJ/Fiji with the free open source SIM image reconstruction plugin fairSlM.

### REFERENCES

1. Liu, D. et al. Three-dimensional controlled growth of monodisperse sub-50 nm heterogeneous nanocrystals. Nat. Commun. 7, 10254 (2016).
2. Duong, H. T. T. et al. Systematic investigation of functional ligands for colloidal stable upconversion nanoparticles. RSC Adv. 8, 4842-4849 (2018).

1.-23. (canceled)

24. A method for performing a multiplex assay, the multiplex assay including using, as probes, a plurality of upconverting nanoparticles (UCNPs) having luminescence profiles, wherein the luminescence profiles possess different rising times, peak moments and/or decay times manipulated through an interfacial energy migration (IEM) process, and the probes are distinguished from one another based on their differing rising times, peak moments and/or decay times.

25. (canceled)

26. A method for preparing a library of spectrally distinct upconverting nanoparticles (UCNPs) comprising:
(a) providing a plurality of different classes of UCNPs, wherein each different class of UCNP has a luminescence profile possessing distinct rising times, peak moments and/or decay times manipulated through a interfacial energy migration (IEM) process;
(b) varying one or more of the following parameters of the UCNPs within each class, so as to provide the library of spectrally distinct UCNPs:
core size of the UCNPs;
concentrations of emitter ions and sensitizer ions in the core;
thickness of a sensitization layer;
concentration of sensitizer ions in the sensitization layer; and
presence or absence of a passivation layer.

27. (canceled)

28. (canceled)

29. The method of claim 26, wherein the different classes of UCNPs are classes of UCNPs having different combinations of activators and/or sensitizers.

30. The method of claim 26, wherein the UCNPs comprise one or more of: neodymium, ytterbium, thulium, erbium, lanthanum, cerium, praseodymium, neodymium, promethium, samarium, europium, gadolinium, terbium, dysprosium, holmium, lutetium, scandium and yttrium.

31. The method of claim 26, wherein the UCNPs comprise neodymium, ytterbium, thulium and/or erbium.

32. The method of claim 26, wherein the UCNPs contain a host material selected from the group consisting of:
an alkali fluoride selected from the group consisting of NaGdF₄, Ca₂F, NaYF₄, LiYF₄, NaLuF₄ LiLuF₄, and KMnF₃,
an oxide which is Y₂O₃ and
an oxysulfide.

33. (canceled)

34. The method of claim 26, wherein the plurality of different classes of UCNPs includes at least one class having core-multi-shell UCNPs, wherein the core-multi-shell UCNPs comprise a core, a migration layer and a sensitization layer.

35. (canceled)

36. The method of claim 34, wherein:
the migration layer comprises Yb³⁺, and/or
the sensitization layer comprises Yb³⁺ and Nd³⁺, and/or
the core comprises: Yb³⁺, Er³⁺ and/or Tm³⁺, Yb³⁺ and Er³⁺, or Yb³⁺ and Tm³⁺.

37. (canceled)

38. (canceled)

39. (canceled)

40. The method of claim 26, wherein the plurality of different classes of UCNPs includes the following: core-multi-shell β-NaYF₄: Nd³⁺, Yb³⁺, Tm³⁺ UCNPs, core-multi-shell β-NaYF₄: Nd³⁺, Yb³⁺, Er³⁺ UCNPs and β-NaYF₄: Yb³⁺, Tm³⁺ UCNPs.

41. The method of claim 26, wherein the UCNPs have a coefficient of variation (CV) value less than about 15%, or less than about 10%, or less than about 5%.

42. (canceled)

43. (canceled)

44. (canceled)

45. (canceled)

46. (canceled)

47. The method of claim 24, wherein the UCNPs comprise one or more of: neodymium, ytterbium, thulium, erbium, lanthanum, cerium, praseodymium, neodymium, promethium, samarium, europium, gadolinium, terbium, dysprosium, holmium, lutetium, scandium and yttrium.

48. The method of claim 24, wherein the UCNPs comprise neodymium, ytterbium, thulium and/or erbium.

49. The method of claim 24, wherein the UCNPs contain a host material selected from the group consisting of:
an alkali fluoride selected from the group consisting of NaGdF₄, Ca₂F, NaYF₄, LiYF₄, NaLuF₄, LiLuF₄, and KMnF₃,
an oxide which is Y₂O₃, and
an oxysulfide.

50. The method of claim 24 wherein the UCNPs are core-multi-shell UCNPs, wherein the core-multi-shell UCNPs comprise a core, a migration layer and a sensitization layer.

51. The method of claim 24, wherein:
the migration layer comprises Yb³⁺, and/or
the sensitization layer comprises Yb³⁺ and Nd³⁺, and/or
the core comprises: Yb³⁺, Er³⁺ and/or Tm³⁺, Yb³⁺ and Er³⁺ or Yb³⁺ and Tm³⁺.

52. The method of claim 24, wherein the UCNPs are selected from the group consisting of:
core-multi-shell β-NaYF₄: Nd³⁺, Yb³⁺, Tm³⁺ UCNPs,
core-multi-shell β-NaYF₄: Nd³⁺, Yb³⁺, Er³⁺ UCNPs and
β-NaYF₄: Yb³⁺, Tm³⁺ UCNPs.

53. The method of claim 24, wherein the UCNPs have a coefficient of variation (CV) value less than about 15%, or less than about 10%, or less than about 5%.
